(19) United States
(12) Patent Application Publication
(10) Pub. No.: US 2016/0346565 A1
(43) Pub. Date: Dec. 1, 2016

(54) METHODS AND APPARATUS TO DELIVER THERAPEUTIC NON-ULTRAVIOLET ELECTROMAGNETIC RADIATION TO A BODY SURFACE

(71) Applicants: Nathaniel L.R. Rhodes, Salt Lake City, UT (US); Mitchell D. Barneck, Portland, OR (US); James P. Allen, Salt Lake City, UT (US); Martin de la Presa, Salt Lake City, UT (US)

(72) Inventors: Nathaniel L.R. Rhodes, Salt Lake City, UT (US); Mitchell D. Barneck, Portland, OR (US); James P. Allen, Salt Lake City, UT (US); Martin de la Presa, Salt Lake City, UT (US)

(21) Appl. No.: 15/167,915

(22) Filed: May 27, 2016

Related U.S. Application Data
(60) Provisional application No. 62/168,082, filed on May 29, 2015.

(51) Int. Cl. A61N 5/06; A61M 1/00; A61M 27/00; A61F 13/02; A61N 1/36

(52) U.S. Cl. CPC A61N 5/0624; A61F 13/0266; A61N 1/36014; A61M 27/00; A61M 1/0088; A61N 2005/0626; A61N 2005/0645; A61N 2005/0652; A61N 2005/067

(57) ABSTRACT

A flexible, therapeutic wound dressing assembly is provided for placement on or in a patient, to absorb biological fluids, to protect a wound, and to deliver therapeutic electromagnetic radiation (EMR) to the patient. The therapeutic wound dressing assembly comprises a wound dressing with at least an optical layer and an outer protective layer and an EMR delivery system with at least one EMR source that emits non-ultraviolet, therapeutic EMR having intensity sufficient to activate desired therapeutic properties within the patient, at least one electronic module that controls EMR output the EMR sources. The EMR output comprises at least one of wavelength, intensity, fluence, frequency, duty cycle, and treatment pattern.

### RELATED APPLICATIONS

This application claims the benefit of U.S. Provisional Application No. 62/168,082 that was filed May 29, 2015, for an invention titled METHODS AND APPARATUS TO INACTIVATE INFECTIOUS AGENTS ON A BODY SURFACE, which is hereby incorporated in its entirety by this reference.

This application is related to a co-pending application entitled METHODS AND APPARATUS TO INACTIVATE INFECTIOUS AGENTS ON A CATHETER RESIDING IN A BODY CAVITY, U.S. application Ser. No. 13/801,750, filed Mar. 13, 2013.

### TECHNICAL FIELD

The present disclosure generally relates to methods and apparatuses to provide therapeutic doses of non-ultraviolet light to stimulate healthy cell growth and healing and/or sterilizing doses of light to inactivate infectious agents on a body surface. In particular, this disclosure is a medical wound dressing device that utilizes non-ultraviolet therapeutic electromagnetic radiation (EMR) at high enough intensity to reduce or eliminate infectious agents in, on, and around a surgical site, wound, or other open or closed skin laceration and/or to stimulate and enhance healing of the wound. This disclosure also describes the methods to work in conjunction with existing wound care products and drainage tubing.

### BACKGROUND

Surgical site and wound infections have varying degrees of occurrence based on the type of surgery, length of surgery and whether surgery was undertaken laparoscopically. One report stated surgical site infections (SSI) occur in about 15% of cases of clean surgery and 30% of contaminated surgery cases (Bruce. The measurement and monitoring of surgical adverse events. Health Technology Assessment 2001; 5:1-194). Another report suggested that SSIs occur in 1 to 3 patients per 100 who have surgery. (Anderson. Strategies to prevent surgical site infections in acute care hospitals. Infect Control Hosp Epidemiol 2008; 29:S51-S61). In the United States alone SSIs are estimated to cause up to $10 billion in annual medical costs (Thompson. Chasing zero: the drive to eliminate surgical site infections. Annals of Surgery 2011; 254(3):430-6). These infections have also been shown to lead to an increase in hospital stay length, increased utilization of resources, and increased mortality (Cruse. The epidemiology of wound infection. A 10-year prospective study of 62,939 wounds. Surg Clin North Am 1980; 60:27).

A traditional wound dressing is a sterile pad that is applied to a wound site held in place by a wrap or adhesive surface. It is designed to stop bleeding by providing positive pressure to a wound site and to expedite clotting, absorb exudate, and protect from infection through wound isolation. These dressings are often made from cloth, or gauze. However, many applications also use films, gels, foams, hydrocolloids, alginates, hydrogels and polysaccharide pastes, granules and beads also. Antibiotic drug coatings, chemicals, and even electrical stimulation have been attempted in order to provide improved healing properties to wound and surgical sites. (Thomas. “An in-vitro comparison of the physical characteristics of hydrocolloids, hydrogels, foams and alginate/CMC fibrous dressings.” SMTL Rep (2005): 1-24). The use of ultraviolet (UV) light, disinfecting chemicals, drugs, to name a few, have been attempted to reduce the prevalence of infection. Many patents have attempted to utilize UV light to disinfect catheters. Unfortunately, UV light is well known to cause damage to living cells (Riffle. “UV-light-induced signal cascades and skin aging.” Ageing research reviews 1.4 (2002): 705-720). U.S. Pat. No. 6,730,113 and U.S. Pat. No. 8,372,128 focus on use of UV light as a main sterilization source. There are also several applications which use this same harmful UV light (US2011/0106223, US2014/0052054) or attempt to claim very broad wavelength spectrums not suitable for an actual embodiment (US2006/0173514, US 2013//0144364, US2014/0207211). EMR in the range of 380-900 nm has been shown to be effective in killing infectious agents. Research done by a group at the University of Strathclyde shows that light in this range is effective in killing surface bacteria in burn wards without harming the patients (Environmental decontamination of a hospital isolation room using high-intensity light. J Hosp Infect. 2010 November; 76(3):247-51). Published patent application US2010/0246169, written by the members who conducted the study, utilizes ambient lighting to disinfect a large surrounding area. The mechanism proposed by the team suggests that light in this range leads to photosensitization of endogenous porphyrins within the bacteria, which causes the creation of singlet oxygen, leading to the death of the bacteria. (Inactivation of Bacterial Pathogens following Exposure to Light from a 405-Nanometer Light-Emitting Diode Array. Appl Environ Microbiol. 2009 April; 75(7):1 932-7).

### SUMMARY OF THE INVENTION

This disclosure relates to a wound dressing assembly for application onto the surface of a patient's body for delivery of therapeutic electromagnetic radiation (EMR) therapy, the protection from said surface wound, and general absorption of biological fluids which may leak from the wound site. The EMR source provides non-ultraviolet, therapeutic EMR having intensity sufficient to inactivate one or more infectious agents and/or to facilitate healing. The wound dressing or bandage has a sterile fabric layer which comes in contact with the patient's skin and surrounds the wound area directly, an array of EMR optical elements which distribute therapeutic EMR to a wound site, a power source for the EMR, and a flexible waterproof layer which has an adhesive portion which comes into contact with the patient's skin as well as a non-adhesive outer portion.

For the purposes of this disclosure the use of the term “therapeutic” should be understood to mean of or relating to the treatment of disease, including infectious agents, as well as serving or performed to maintain health, including enhancing healthy cell growth. Also, for purposes of this disclosure the use of the term “wound area” should be understood to mean the area in the close vicinity to the wound and/or including the area of traumatized tissue surrounding the wound.

Any suitable power source may be used to activate the EMR source to provide the non-ultraviolet, therapeutic EMR. The power source may provide either direct (DC) or alternating current (AC) power. With AC power, the power source may be remote from the wound dressing and connected by wires as is customary. With DC power, the power may be either self-contained within the wound dressing such as a battery, or may be remote from the wound dressing such as a battery pack or a solar generator that would be connected by wires to the wound dressing.

This disclosure also provides methods and apparatuses for effectively sterilizing the body surface for the area in, on, or around the surrounding wound area. This is done through use of EMR at sufficient intensities capable of inactivation of infectious agents. This EMR source can be from one or more optical elements including a light emitting diode, a semiconductor laser, a diode laser, an electroluminescent wire, and an incandescent or fluorescent light source. The optical element(s) used may be directed toward the wound area of a patient's body either directly or indirectly. Also, the EMR source may be disposed within the wound dressing or it may be remote from the wound dressing and delivering the therapeutic EMR to the wound dressing using a delivery assembly that may include fiber optics or other suitable light conduits and lenses.

This EMR source provides non-ultraviolet, sterilizing EMR having a wavelength in the range of approximately 380 nm to approximately 900 nm. In order to provide sufficient inactivation of infectious species, the light should be of a narrow spectrum and centered around at least one wavelength from the group of several wavelengths including: 400 nm, 405 nm, 415 nm, 430 nm, 440 nm, 455 nm, 470 nm, 475 nm, 660 nm, and 808 nm, wherein each of the several wavelengths has an intensity sufficient to inactivate one or more infectious agents. Because the intensity and power of the light emitted is important for the inactivation of infectious agents, a range of fluences covering 0.1 J/cm² to 1 kJ/cm² and a range of powers from 0.005 mW to 1 W, and power density range covering 1 mW/cm² and 1 W/cm² are of are particularly suitable for providing the intensity and power required to inactivate infectious agents for a wound dressing assembly.

Also of interest to the wound dressing assembly of the present disclosure is the use of different wavelengths between 532 nm and 1064 nm for stimulating tissue healing properties. Exemplary wavelengths have demonstrated desirable tissue healing properties, including those wavelengths centered about 633 nm, 808 nm, and 830 nm. Doses ranging from 0.09 to 90 J/cm² have been demonstrated to be effective, with the predominating values from 1 to 5 J/cm². However, doses 150 J/cm² are of particular interest for the applications contemplated by this disclosure.

For each exemplary embodiment, the wound dressing assembly and method for disinfection and/or healing enhancement could be utilized in an adjustable or predetermined duty cycle. Moreover, more than one wavelength could be delivered simultaneously, alternatingly, or pattern alternatingly. If treatments began immediately after sterile procedure was initiated, skin or device related infections may be inhibited. This includes device-related biofilm growth.

The optical elements of the EMR source of an exemplary embodiment may be arranged in an array or in sub-arrays to accomplish the delivery of therapeutic EMR in a versatile manner. For example, space between each optical element may be filled with a material that absorbs or wicks biological fluids away from the wound so that the therapeutic EMR is compromised by passing through an accumulation of such biological fluids. Also, although some optical elements are capable of delivering multiple wavelengths simultaneously, it may be advantageous to deliver the therapeutic EMR by using sub-arrays of optical elements where each sub-array is delivering differing therapeutic EMR at different wavelengths, intensity, duty cycles either simultaneously or alternatingly.

In one exemplary embodiment, a flexible wound dressing is applied to the surgical site of a patient's body. A sterilizing EMR source is activated and irradiated onto the wound area of the skin surface to inactivate bacterial and fungal agents.

In another exemplary embodiment, a flexible wound dressing is applied to a surgical site where wound drainage tubes have been placed. The wound dressing has a perforated center hole or an aperture that accommodates the passage of the drainage tube through the wound dressing without compromising the delivery of the therapeutic EMR. A perforated line may extend from the perforated center hole or aperture to the periphery of the wound dressing to facilitate the application of the wound dressing around a drainage tube that is already in place, so that the drainage tube need not be disturbed when applying the wound dressing.

In another exemplary embodiment, the flexible wound dressing comprises a fabric wrap attached to at least one portion of the EMR delivery system. The wound dressing may be applied to the wound area on the surface of the skin, the EMR activated, and wrap may be wrapped around the body or limb of the patient to provide pressure to the skin surface in addition to the therapeutic EMR.

Where the EMR source is disposed within the wound dressing, heat might become an issue over time. Another exemplary embodiment addresses heat issues, if any, by providing a heat dissipation layer within the wound dressing to draw the heat away from the patient. Such dissipation layer may comprise any suitable material and may operate using conduction, convection, or a combination of both. For example, heat conductive materials such as aluminum, copper, tungsten, silicon carbide, graphite, diamond, carbon fibers, and other conductive materials may be used. Also, convection fluids such as water, saline solutions, air, and heat absorbing gases may be circulated through a manifold in the heat dissipation layer and to and through a heat exchanger. For optimum heat dissipation, the convection manifold may be surrounded by a heat conductive material so that heat is drawn away from the wound dressing by both conduction and convection.

In yet another exemplary embodiment, the flexible wound dressing could be inserted into a female support device for post breast surgery wound sterilization. This embodiment would not need to contain an adhesive layer but instead would comprise an elastic or band to provide support and keep the therapeutic EMR close to the surgical site.

In still another exemplary embodiment, the flexible wound dressing could be inserted into surgical drapes to provide sterilizing irradiation to the surgical site during the time of surgery. This embodiment would not need to contain an adhesive layer but instead would comprise an elastic or band to provide support and keep the EMR close to the surgical site.

In another exemplary embodiment, the EMR originating source is not contained within the wound dressing, yet it is coupled to an external EMR source which delivers through optical elements to the wound dressing. This embodiment would include an attachment point for optical coupling into the wound dressing. It may also interface with an existing wound drainage tubing to provide sterilizing radiation into said drainage tubing.

Still another exemplary embodiment of a flexible wound dressing may combine negative pressure therapy with the therapeutic EMR by combining the EMR delivery system with a vacuum pump to deliver negative pressure to the wound area either simultaneously, intermittently, and/or alternatingly with the therapeutic EMR without unnecessarily removing and replacing the wound dressing.

### BRIEF DESCRIPTION OF THE DRAWINGS

These and other features and advantages of the present disclosure will become more readily appreciated by referring to the following detailed description of exemplary embodiments when considered in connection with the accompanying drawings, which are not necessarily drawn to scale. It will be understood that said drawings depict exemplary embodiments and, therefore, are not to be considered as limiting the scope with regard to other embodiments which the invention is capable, wherein:

FIG. 1 is a schematic view of an exemplary wound dressing as applied to a patient showing the application having a drainage tube placed through the wound dressing;

FIG. 2 is an, exploded cross-sectional view of another exemplary wound dressing for a knee wound, showing the direction of the EMR emission;

FIG. 3a is an exploded view of an exemplary wound dressing, showing a semi-translucent fabric layer, a flexible EMR source layer, and an outer protective layer;

FIG. 3b is an exploded view of another exemplary wound dressing, showing a higher density of EMR sources creating a potentially larger power density and/or multiple dose emission for treatments;

FIG. 4a is an exploded view of yet another exemplary wound dressing, showing a self-contained power source (such as a battery) and an exemplary switch capable of engaging the EMR;

FIG. 4b is an exploded view of yet another exemplary wound dressing, showing a self-contained power source (such as a battery), an exemplary switch capable of engaging the EMR, and a heat dissipation layer;

FIG. 5a is a plan view of an exemplary array of EMR sources coupled together to emit EMR onto a skin surface to prevent infections from forming and/or to promote healing;

FIG. 5b is a plan view of another exemplary array of EMR sources coupled together to emit EMR onto a skin surface to prevent infections from forming and/or to promote healing, and showing a perforated slit and opening to facilitate the inclusion of wound drainage tubing, catheters, syringes, and the like;

FIG. 6 is a perspective embodiment of a non-traditional wound dressing in a support bra for chest applications, showing EMR sources located inside of cups over the surgical/wound sites to prevent infection and/or to promote healing;

FIG. 7 is a depiction of exemplary duty cycle waves showing the various exemplary duty cycles and component terminology;

FIG. 8 is an exemplary circuit diagram for an exemplary electronic module for an exemplary wound dressing;

FIG. 9 is a schematic view of an exemplary wound dressing as applied to a patient showing the application of negative-pressure therapy through a pressure conduit placed through the wound dressing and connected to a remotely located vacuum pump;

FIG. 10 is a schematic view of an exemplary wound dressing as applied to a patient showing the combination application of therapeutic EMR and negative-pressure therapy through a delivery conduit connected to a remotely located EMR source/vacuum pump combination unit;

FIG. 11a is a perspective view of an exemplary embodiment showing a roll of wound dressings capable of wrapping around a body part;

FIG. 11b is a perspective, partially exploded view of the exemplary embodiment roll of wound dressings of FIG. 11a;

FIG. 11c is a perspective view of an exemplary embodiment showing a roll of wound dressings as depicted in FIGS. 11a and 11b wherein the roll is unrolled partially to show an extended non-adhesive tail for wrapping around a body part;

FIG. 12 is a perspective view of an exemplary embodiment showing multiple individual wound dressings in a roll for packaging purposes;

FIG. 13a is a schematic view of an exemplary wound dressing with self-contained controls on a roll showing an initial stage of as applying the exemplary wound dressing to a patient;

FIG. 13b is a schematic view of an exemplary wound dressing with self-contained controls on a roll of FIG. 13b showing the final stage of as applying the exemplary wound dressing to a patient so that the controls are not obscured; and

FIG. 14 is a schematic view of an exemplary wound dressing as wrapped around a patient showing the combination application of therapeutic EMR and negative-pressure therapy through a delivery conduit connected to a remotely located EMR source/vacuum pump combination unit.

### REFERENCE NUMERALS

| | |
|---|---|
| wound dressing assembly 10 | patient 12 |
| wound dressing 14 | optical layer 16 |
| outer protective layer 18 | EMR delivery system 20 |
| EMR source 22 | non-ultraviolet, therapeutic EMR 24 |
| | (EMR output) |
| electronic module 26 | power supply 28 |
| drainage tube 30 | wound area 32 |
| knee 34 | non-adhesive outer layer 36 |
| an inner adhesive layer 38 | arrows 40 |
| central adhesive area 42 | array 44 |
| interstices 46 | self-contained power source 48 |
| general switch 50 | On switch 52 |
| Off switch 54 | heat dissipating layer 56 |
| ridges 58 | electrical junctures 60 |
| perforated slit 62 | aperture 64 |
| periphery 66 | support bra 68 |
| cup(s) 70 | diffuser 72 |
| adjustable straps 74 | duty cycle waves 76 |
| topmost duty cycle wave 78 | pulse width 80 |
| period 82 | interpulse interval 84 |
| upper middle duty cycle wave 86 | lower middle duty cycle wave 88 |
| lowermost duty cycle wave 90 | digital potentiometer 92 |
| microcontroller 94 | sensor(s) 96 |
| display/user interface 98 | pressure conduit 100 |
| vacuum pump 102 | delivery conduit 104 |

EMR source/vacuum pump combination unit 106

| | |
|---|---|
| roll 108 | compression bandage wrap 110 |
| gauze bandage layer 112 | non-adhesive tail(s) 114 |
| perforated strip 116 | |

### DETAILED DESCRIPTION

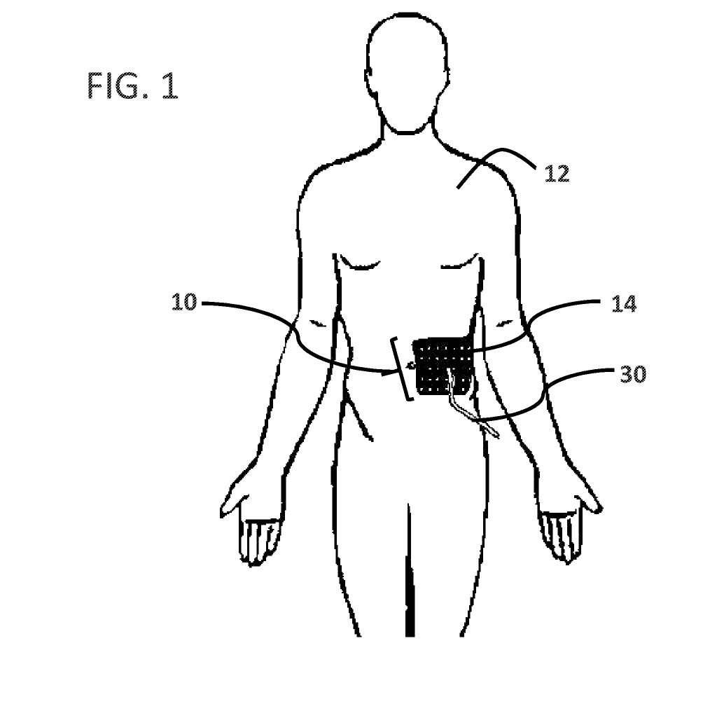

Various exemplary embodiments of the present disclosure are described more fully hereafter with reference to the accompanying drawings. These drawings illustrate some, but not all of the embodiments of the present disclosure. It will be readily understood that the components of the exemplary embodiments, as generally described and illustrated in the Figures herein, could be arranged and designed in a wide variety of different configurations. Thus, the following more detailed description of the exemplary embodiments of the apparatus, system, and method of the present disclosure, as represented in FIGS. 1 through 14, is not intended to limit the scope of the invention, as claimed, but is merely representative of exemplary embodiments.

The phrases “connected to,” “coupled to” and “in communication with” refer to any form of interaction between two or more entities, including mechanical, electrical, magnetic, electromagnetic, fluid, and thermal interaction. Two components may be coupled to each other even though they are not in direct contact with each other. The term “abutting” refers to items that are in direct physical contact with each other, although the items may not necessarily be attached together.

The word “exemplary” is used exclusively herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Traditional wound dressings are sterile pads that are applied to a wound site and held in place by a wrap or adhesive surface. Such wound dressings are designed to stop bleeding by providing positive pressure to a wound site and to expedite clotting, absorb exudate, and protect from infection by isolating the wound. These dressings are often made from cloth, or gauze. However, many applications also use films, gels, foams, hydrocolloids, alginates, hydrogels and polysaccharide pastes, granules and beads. In recent years, antibiotic drug coatings, chemicals, and even electrical stimulation have been attempted to provide improved healing properties to wound and surgical sites. However, despite those efforts, infections at wound sites remain a significant medical problem, particularly for longer-term patients at health institutions.

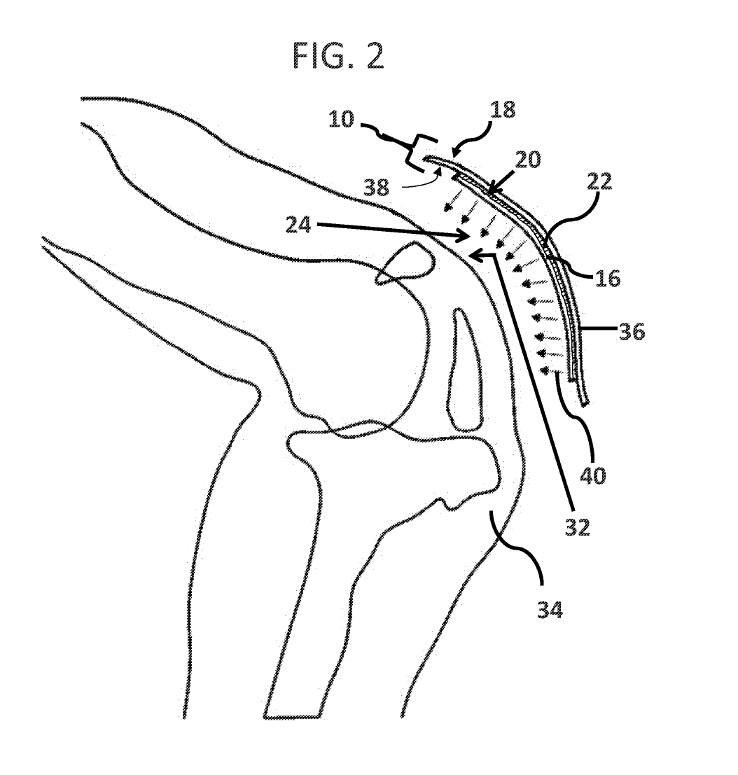

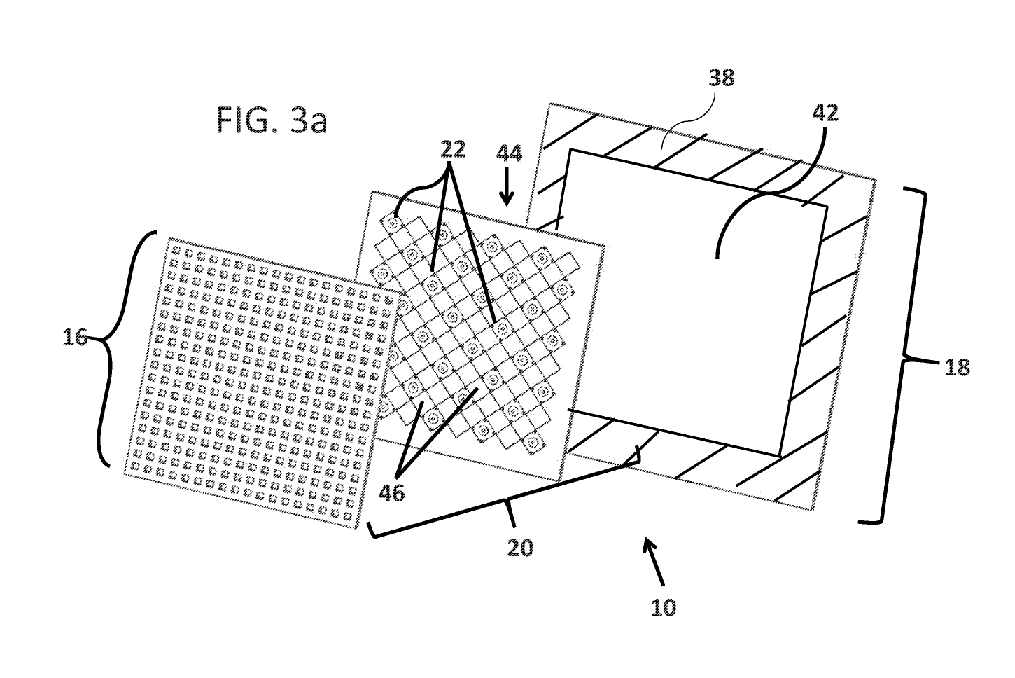

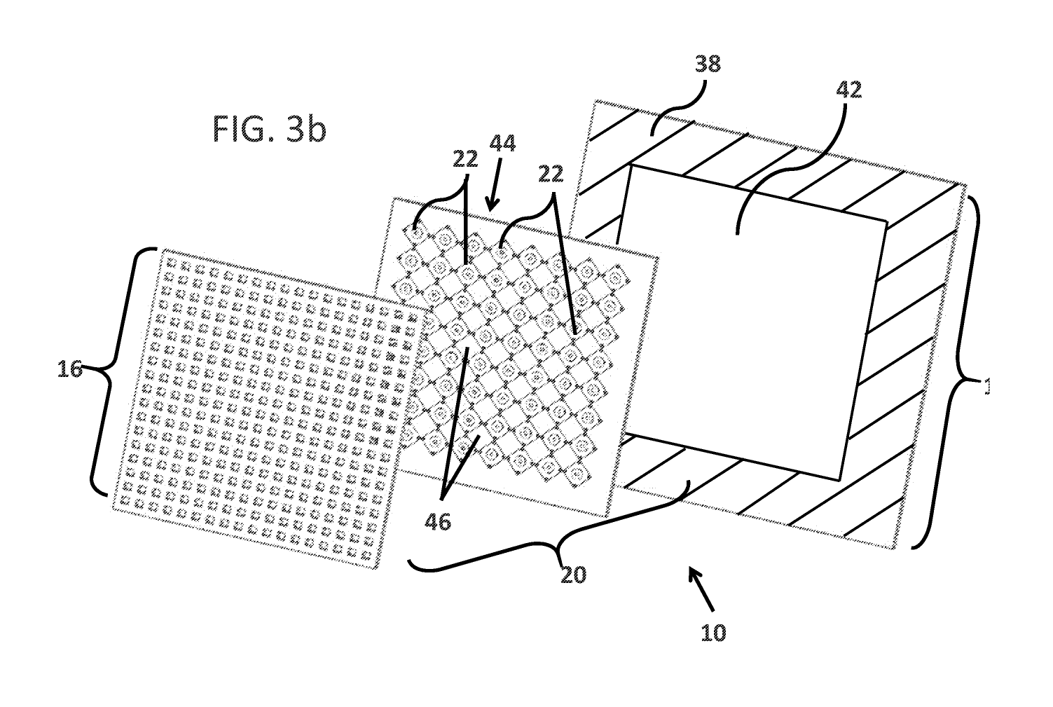

Referring now to FIGS. 1 through 3b of the present disclosure, a flexible, exemplary therapeutic wound dressing assembly 10 is shown. FIG. 1 is a schematic view of an exemplary wound dressing assembly as applied to a patient 12 showing the application having a drainage tube 30 placed through the wound dressing 14. FIG. 2 is an, exploded cross-sectional view of another exemplary wound dressing assembly 10 for a knee 34 wound, showing the direction of the EMR output 24. FIG. 3a is an exploded view of an exemplary wound dressing assembly 10 showing a semi-translucent fabric optical layer 16, a flexible EMR delivery system 20 layer, and an outer protective layer 18. FIG. 3b is an exploded view of another exemplary wound dressing assembly 10 showing a higher density of EMR sources 22 creating a potentially larger power density and/or multiple dose emission for treatments.

The therapeutic wound dressing assembly 10 is designed for placement on or in a patient 12, to absorb biological fluids (not shown), to protect a wound (not shown), and to deliver therapeutic electromagnetic radiation (EMR) to the patient 12. The therapeutic wound dressing assembly 10 comprises a wound dressing 14 having at least two layers, an optical layer 16 and an outer protective layer 18, and an EMR delivery system 20.

The optical layer 16 may have at least an optical portion comprising at least one of an optically clear material, a translucent material, and a semi-opaque material, each such material allows the therapeutic EMR to pass through with minimal optical obstruction. For example, the optical layer 18 may be a film, a fabric such as cloth or gauze, a combination of or multiple layers of film and/or fabric, or any other suitable material, some of which may be mentioned in this disclosure although not called out in this paragraph.

The outer protective layer 18 of the exemplary flexible wound dressing assembly 10 is depicted as applied to a patient 12. The outer protective layer 18 may be made of any suitable material that assists in isolating the wound. Many materials have been used as an outer layer for bandages, some to assist with positive pressure application, breathability, and adherence to the patient 12. It is advantageous to maximize the breathability for the wound without subjecting the wound to infectious agents. Also, it may be advantageous to have a portion of the outer protective layer 18 and all subtending layers to be transparent so that the medical personnel may be able to observe and determine when treatment has been completed. This application contemplates the use of all known materials that have been used as an outer layer for a bandage.

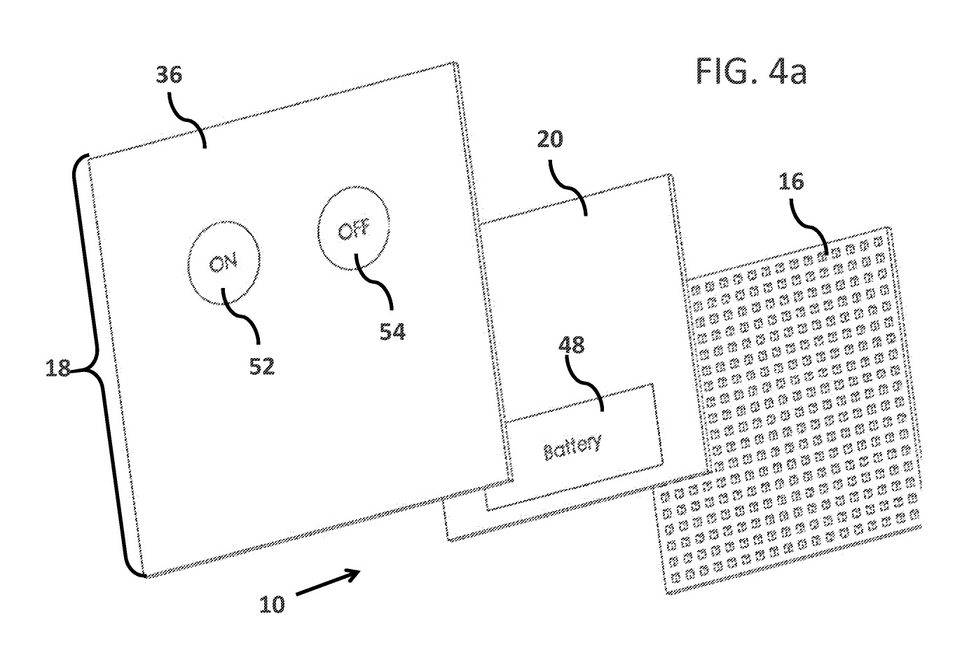

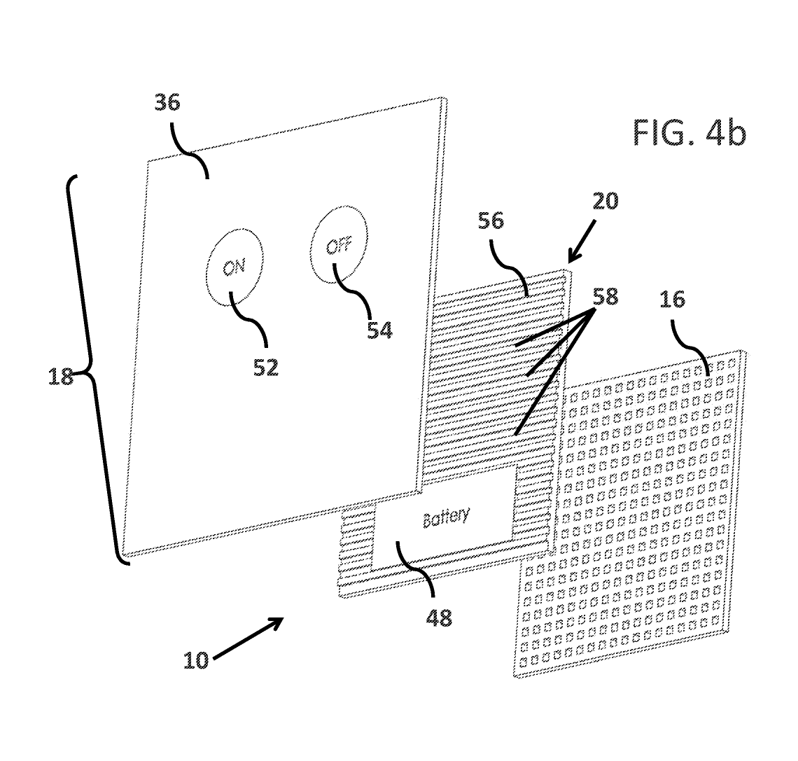

The EMR delivery system 20 comprises at least one EMR source 22 that emits non-ultraviolet, therapeutic EMR 24 (FIG. 2) (also referred to EMR output 24) having intensity sufficient to activate desired therapeutic properties within the patient 12, at least one electronic module 26 (see FIGS. 4a and 4b) that controls EMR output 24 from at least one EMR source 22, and a power supply 28 for the EMR source 22 (not shown, see FIGS. 4a, 4b, 8-10, and 14). The EMR output 24 has various characteristics which may be controlled by the electronic module 26 including but not limited to wavelength, intensity, fluence, frequency, duty cycle, and treatment pattern;

For purposes of this disclosure and the claims, the power supply 28 may be any suitable power supply 28, including a self-contained battery, a battery pack (charged by any means such as solar, wind, AC, etc.), a power cord that may be plugged into a generator, a power outlet, or attached to a battery, and anything that causes the EMR source to activate.

As depicted in FIG. 1, the exemplary flexible wound dressing assembly 10 also has a drainage tube 30 extending therefrom for draining biological fluids from the covered wound area 32 (not shown, see FIG. 2)). The drainage tube 30 may be inserted through the flexible wound dressing assembly 10 by a medical provider or the flexible wound dressing assembly 10 may be manufactured with a drainage tube 30 disposed through the wound dressing 14.

The exemplary flexible wound dressing assembly 10 depicted in FIG. 1 permits for fluid exudate to be removed from the wound while non-ultraviolet, therapeutic EMR 24 may be applied to the wound area 32 (not shown, see FIG. 2). Hence, the exemplary flexible wound dressing assembly 10 may eliminate or significantly reduce the infectious agents at or near the wound site and/or may promote healthy cell growth and healing.

Another exemplary flexible wound dressing assembly 10 is depicted in FIG. 2, showing in an exploded sectional view the knee 34 of a patient 12 with the exemplary flexible wound dressing assembly 10 about to be applied to the knee 34. The exemplary flexible wound dressing assembly 10 depicted comprises an EMR delivery system 20 for providing non-ultraviolet, therapeutic EMR 24 via EMR sources 22, an optical layer 16 (such as a semi-translucent fabric) that allows therapeutic EMR 24 to pass through with minimal optical obstruction (non-occlusive), and an outer protective layer 18. The outer protective layer 18 (as best seen in FIGS. 3a and 3b) may comprise a non-adhesive outer layer 36, and an inner adhesive layer 38. The direction of the EMR output 24 is indicated by the arrows 40 pointing towards the wound area 32. In should be understood that the EMR output 24 may be directed either directly or indirectly toward the wound area 32. FIG. 2 shows direct EMR output 24; however, a reflective layer (not shown) may be positioned behind the EMR source(s) 22 so that the EMR output 24 may reflect off of the reflective layer before reaching the wound area 32.

Therapeutic EMR 24 having a sterilizing effect shall be defined as EMR output 24 manifested as light emitted within in a range from approximately 380 nm to 900 nm having a high intensity sufficient to inactivate one or more infectious agents.

FIG. 3a is an exploded view of an exemplary flexible wound dressing assembly 10. This exemplary embodiment of the flexible wound dressing assembly 10 comprises an EMR delivery system 20 internal to the wound dressing assembly 10 for providing non-ultraviolet, therapeutic EMR 24 via EMR sources 22 with a semi-translucent fabric, optical layer 16 that may allow therapeutic EMR 24 to pass through with minimal optical obstruction and also may absorb biological samples; an outer protective layer 18 comprising a non-adhesive outer layer 36 (see FIGS. 4a and 4b) and an inner adhesive layer 38. The inner adhesive layer 38 also has a central adhesive area 42 for securing the EMR delivery system 20 to the outer protective layer 18.

FIG. 3b is an exploded view of another exemplary embodiment of a flexible wound dressing assembly 10, differing from the flexible wound dressing assembly 10 of FIG. 3a in that the EMR delivery system 20 has a higher density of EMR sources 22. This exemplary embodiment contains an EMR delivery system 20 with a higher density (double) of EMR sources 22 creating a potentially larger power density for treatments. Like the previous embodiment, this exemplary embodiment comprises an EMR delivery system 20 for providing non-ultraviolet, therapeutic EMR 24 via EMR sources 22 with a semi-translucent fabric, optical layer 16 capable of allowing therapeutic EMR 24 to pass through with minimal optical obstruction and also may absorb biological samples; an outer protective layer 18, comprising a non-adhesive outer layer 36 and an inner adhesive layer 38. The inner adhesive layer 38 also contains a central adhesive area 42 for securing the EMR delivery system 20 to the outer protective layer 18.

As shown in FIGS. 3a and 3b, the EMR sources 22 are arranged in a grid-like array 44 so to provide the therapeutic EMR 24 over a broad wound area. Between each of the EMR sources 22 are interstices 46 that may be openings or openings that are filled with a fluid absorbing material (like gauze, for example) or a wicking material that will draw the biological fluids away from obstructing the EMR output 24.

It should also be understood that the array 44 of EMR sources 22 may comprise sub-arrays of EMR sources 22 wherein each sub-array may have at least one differing characteristic from each other sub-array. In that way, several different wavelengths, intensities, fluences, frequencies, duty cycles, and treatment patterns may be provided from a single EMR delivery system 20. By using sub-arrays or distinct EMR sources 22, another exemplary embodiment may have a first EMR source 22 for delivering a first therapeutic EMR 24 to a first location on the patient 12 and a second EMR source 22 for delivering a second therapeutic EMR 24 to a second location on the patient 12, where the first therapeutic EMR 24 is different from the second therapeutic EMR 24 and the first location is different from the second location.

It should also be understood that the EMR delivery system 20 may be detached from the optical layer 16 so a soiled optical layer 16 may be replaced by a fresh optical layer 16 and the EMR delivery system 20 may be reused. Of course, reusing the EMR delivery system 20 may require sterilization by any one of several suitable sterilization techniques using ethylene oxide, formaldehyde, and autoclave, gamma radiation, electron-beam radiation, and any other acceptable sterilization method. The EMR delivery system 20 is described more fully hereinafter.

Referring now to FIGS. 4a and 4b, an exploded view of yet another exemplary the flexible wound dressing assembly 10 having an EMR system 20 with a self-contained power source 48 is depicted and is shown from a viewpoint directed in opposite direction from the viewpoint of FIGS. 3a and 3b. This exemplary embodiment comprises of an EMR system 20 which may include a power supply 28 such as a battery as a self-contained power source 48. Additionally, the outer protective layer 18 may be integrated with the electronic module 26 (not shown) and/or may have a general switch 50 (e.g., bi-modal or multi-modal) by which the EMR delivery system 20 may be toggled on or off by depressing either an On switch 52 or an Off switch 54. Of course, it should be understood that the control of the EMR output 24 may be as simple as ON/Off or it may also involve the electronic module 26 and be controlled in a manner that selectively and/or incrementally increases/decreases EMR output 24 intensity or selectively and/or incrementally increases/decreases frequency of the light emitted by a slide switch, a dial or the like, or control may be provided to control any of the characteristics mentioned herein. Again, the semi-translucent fabric, optical layer 16 may allow therapeutic EMR 24 to pass through with minimal optical obstruction and may absorb biological samples.

FIG. 4b is similar to the exemplary embodiment depicted in FIG. 4a except that the backside of the EMR delivery system 20 has a heat dissipating layer 56 with ridges 58 to create additional surface area for more efficient heat dissipation. Otherwise, the layers may be substantially the same as the layers described with respect to FIG. 4a. Such dissipation layer 56 may comprise any suitable material and may operate using conduction, convection, or a combination of both. For example, heat conductive materials such as aluminum, copper, tungsten, silicon carbide, graphite, diamond, carbon fibers, and other conductive materials may be used. Also, though not shown in FIG. 4b, convection fluids such as water, saline solutions, air, and heat absorbing gases may be circulated through a manifold embedded within the heat dissipation layer 56 and flowing to and through a heat exchanger (not shown). For optimum heat dissipation, the convection manifold may be surrounded by a heat conductive material so that heat is drawn away from the wound dressing by both conduction and convection. Although the use of convection is not specifically shown, armed with this disclosure, those skilled in the art could readily construct a convection system using a manifold and a combination convection/conduction system.

Of course, it also should be understood that the power source 48 need not be self-contained within the flexible wound dressing assembly 10, but that a person of skill in the art, armed with this disclosure, could easily determine how power may be supplied to the EMR delivery system 20 and EMR sources 22 from a power supply 28 remote from the flexible wound dressing assembly 10.

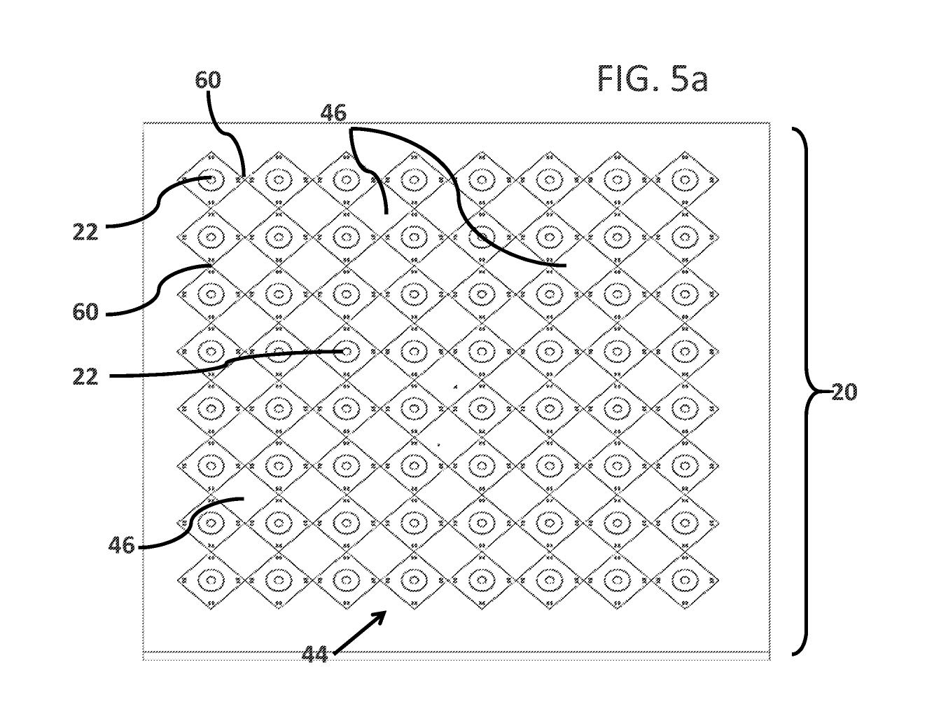

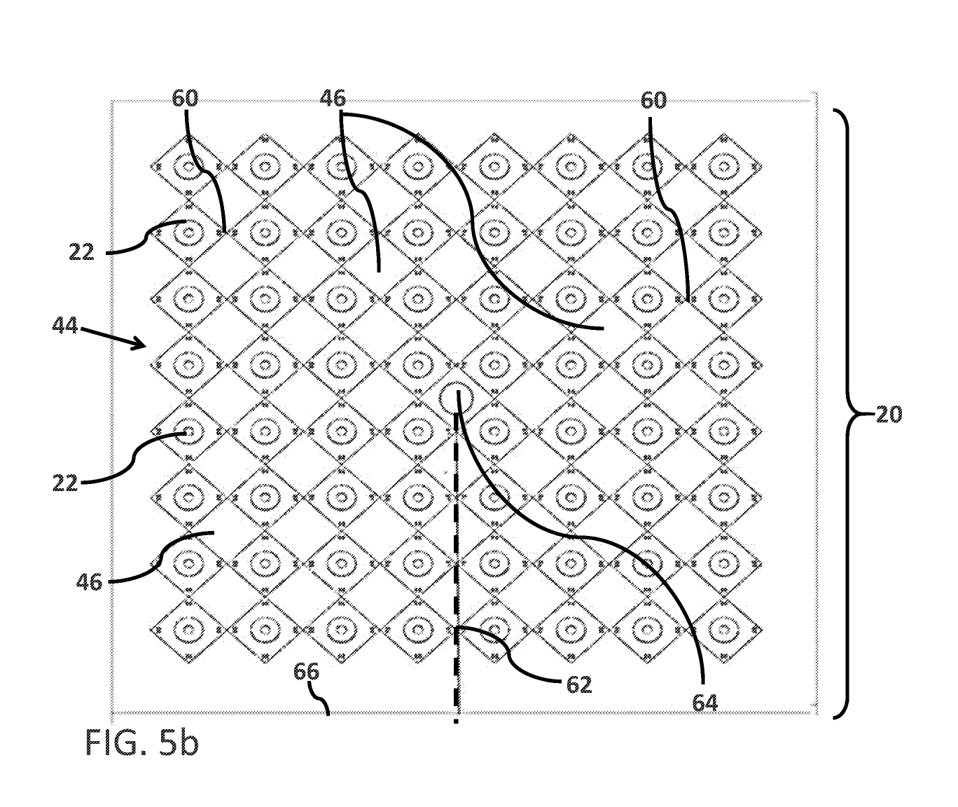

Turning to FIGS. 5a and 5b, FIG. 5a is a plan view of an exemplary EMR delivery system 20 comprising an array 44 (and/or sub-arrays) of EMR sources 22 electrically coupled together at electrical junctures 60. Upon activation, the EMR sources 22 may illuminate and emit EMR output 24 onto a body surface. This array 44 of EMR sources 22 provides sufficient intensity and fluence of light to eliminate infectious agents residing on a body surface and/or to stimulate healthy cell growth and healing. The interstices 46, as mentioned above, are openings that may be left open to help dissipate heat or may be filled with an absorbing or wicking material to carry biological fluids away from obstructing the EMR output 24.

FIG. 5b is a plan view of another exemplary EMR system 20 containing multiple EMR sources 22 electrically coupled together at electrical junctures 60. Again, upon activation, the EMR sources 22 may illuminate and emit EMR output 24 onto a body surface. The array 44 of EMR sources 22 provides sufficient intensity and fluence of light to eliminate infectious agents residing on a body surface and/or to stimulate healthy cell growth and healing. This embodiment differs from the embodiment depicted in FIG. 5a in that it has a perforated slit 62 and an aperture 64 in a portion of the flexible wound dressing assembly 10 to facilitate the insertion of wound drainage tubing 30, catheters, probes, and the like through the flexible wound dressing assembly 10. The aperture 64 may be perforated so that the aperture 64 remains closed until the perforation is torn free to open the aperture 64. The perforated slit 62 extends between the opening 64 and the periphery 66 of the wound dressing 14 so that when torn free the slit created may facilitate the application of the wound dressing 14 around a drainage tube 30, a catheter, a probe, or the like that is already in place, so that the drainage tube 30 or other protrusions need not be disturbed when applying the wound dressing 14.

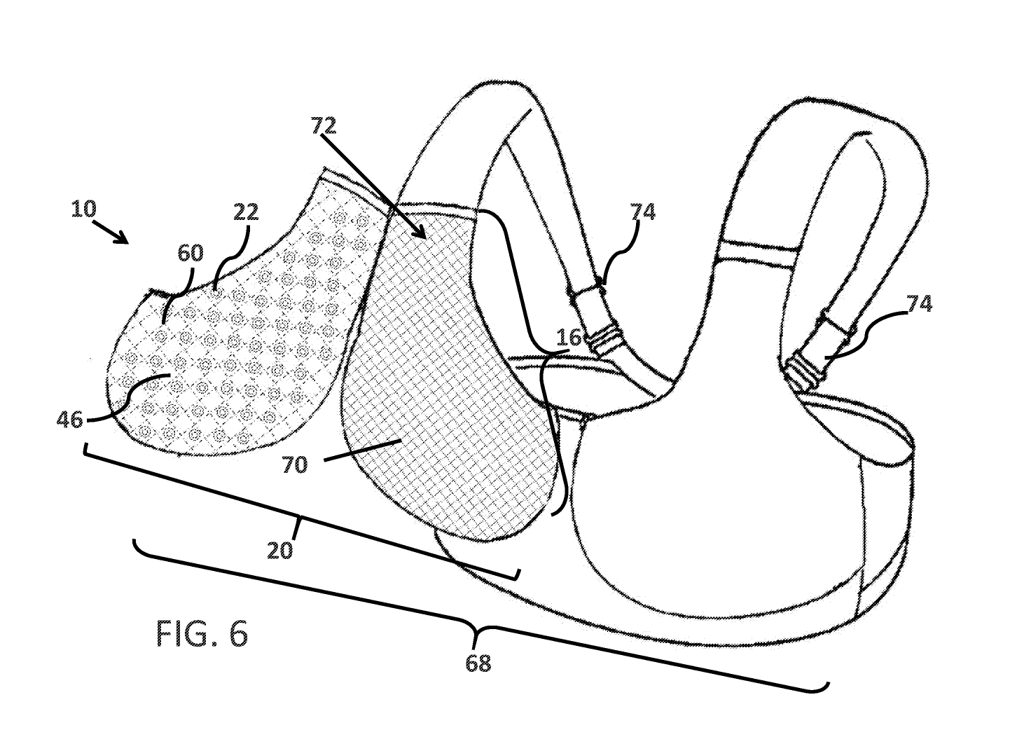

Referring now to FIG. 6, a perspective view of an exemplary embodiment of a non-traditional type of flexible wound dressing assembly 10 fashioned as support bra 68 for chest applications. This embodiment is designed for patients 12 who have had breast and chest wounds or surgery. The non-ultraviolet, therapeutic EMR delivery system 20 is located inside of cups 70 over the surgical/wound sites. In addition to the EMR delivery system 20, this embodiment contains an optical layer 16 within the cup 70 that serves as a diffuser 72 that diffuses the therapeutic EMR 24 such that the therapeutic EMR 24 is distributed relatively evenly over the surface of the wound area 32 covered by the wound dressing assembly 10. The optical layer 16 allows therapeutic EMR 24 to pass through with minimal optical obstruction and may absorb biological samples. An outer protective layer 18 is also provided to nest over the diffuser 72 and optical layer 16. The EMR delivery system 20 has a plurality of EMR sources 22 for providing non-ultraviolet, therapeutic EMR 24. In addition, this embodiment also contains adjustable straps 74 to aid in positioning the EMR delivery system 20. It should be understood that the non-traditional type of flexible wound dressing assembly 10 depicted in FIG. 6 in merely exemplary and that other non-traditional types of flexible wound dressing assemblies 10 are contemplated. For example, specialty wraps for necks, shoulders, elbows, ribs, backs, hips, knees, ankles, feet, and the like may be equipped with EMR delivery systems 20 with EMR sources 22.

Also, it should be understood that the diffuser 72 may be its own layer, may be one or more layers of gauze serving as the optical layer 16, or may be a diffusing film or fabric disposed on or within the optical layer 16 between the EMR sources 22 and the wound area 32.

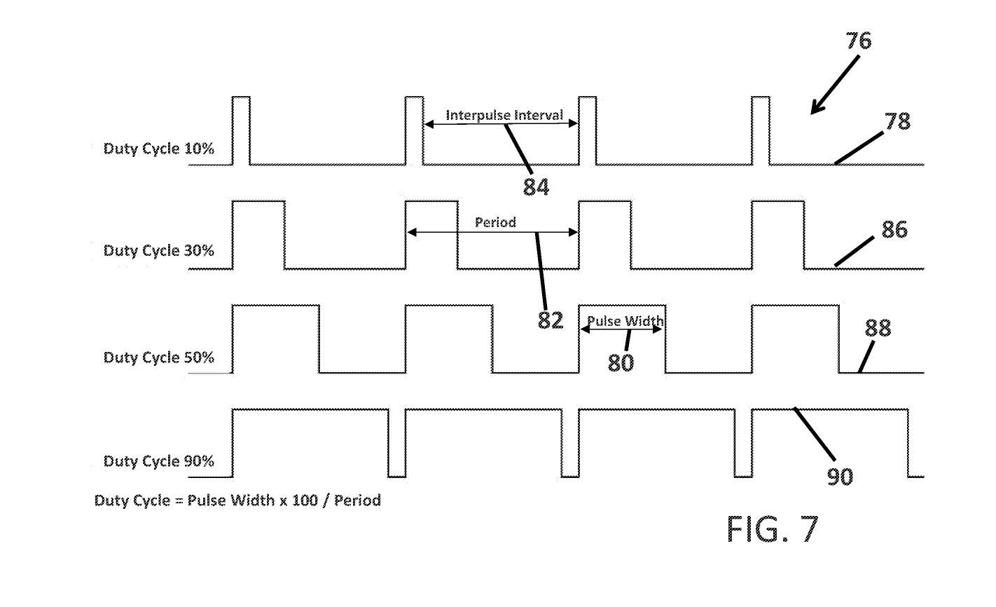

FIG. 7 is a depiction of exemplary duty cycle waves 76 showing the various exemplary duty cycles and component terminology. The topmost duty cycle wave 78 represents a 10% duty cycle, i.e., the therapeutic EMR 24 is active for a pulse width 80 of 10% of each period 82 and the interpulse interval 84 is 90% of the period 82. The upper middle duty cycle wave 86 represents a 30% duty cycle, i.e., the therapeutic EMR 24 is active for the pulse width 80 of 30% of each period 82 and the interpulse interval 84 is 70% of the period 82. The lower middle duty cycle wave 88 represents a 50% duty cycle, i.e., the therapeutic EMR 24 is active for the pulse width 80 of 50% of each period 82 and the interpulse interval 84 is 50% of the period 82. The lowermost duty cycle wave 90 represents a 90% duty cycle, i.e., the therapeutic EMR 24 is active for the pulse width 80 of 90% of each period 82 and the interpulse interval 84 is 10% of the period 82. Each of the exemplary duty cycle waves 76 are merely representative of an infinite range of duty cycles that may be selected to provide the appropriate dose of therapeutic EMR 24 for any given patient's 12 needs. The exemplary duty cycle waves 76 depicted are regular period 82 duty cycles, but they be irregular period 82 duty cycles or alternating regular period 82 duty cycles if such duty cycles are selected as an appropriate dose of therapeutic EMR 24 for any given patient's 12 needs. Of course, various exemplary embodiments of the wound dressing assembly 10 of the present disclosure may provide duty cycles that are predetermined, selected to address particular needs, or adjustable to meet changing needs.

By way of example, the therapeutic EMR 24 may be pulsed at a rate of between 0 Hz and 5,000 Hz, have a pulse width 80 of between 10 nanoseconds and 1 second, and have an interpulse interval within a range of 1 nanosecond to 1 second.

With some exemplary embodiments, at least one wavelength of therapeutic EMR 24 comprises a predominant wavelength selected to sterilize one or more target organisms and/or to promote healthy cell growth and healing, the predominant wavelength is selected from a group of wavelengths consisting of wavelengths centered about 400 nm, 405 nm, 415 nm, 430 nm, 440 nm, 455 nm, 470 nm, 475 nm, 660 nm, and 808 nm for sterilizing and is selected from a group of wavelengths consisting of wavelengths centered about 633 nm, 808 nm, and 830 nm for promoting healing. Additionally, in some embodiments the predominant wavelength alternates between a first predominant wavelength and a second predominant wavelength in a selected treatment pattern.

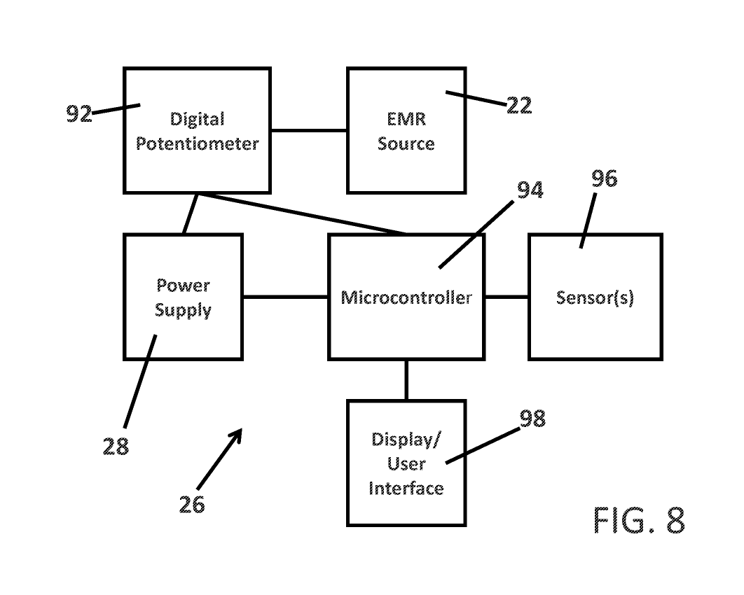

Referring now to FIG. 8, a circuit diagram for an exemplary electronic module 26 for an exemplary wound dressing 14 is depicted. The exemplary electronic module 26 comprises a power supply 28, a digital potentiometer 92, an EMR source 22, a microcontroller 94, one or more sensors 96, and a display/user interface 98. This exemplary electronic module 26 may be housed in a control housing (not shown) that is external to the wound dressing assembly 10 or it may be wholly or partially within one or more layers of the wound dressing 14.

As noted above, the power supply 28 may be an AC or DC power supply. By way of example, the power supply 28 may range from 1V-24V (DC) or it may be 110V or 220V (AC) or whatever voltage is used in the geographic location where it is operated. In some embodiments, a 9V power supply 28 may be accomplished by using coin cell batteries.

The digital potentiometer 92 is a digitally controlled electronic component that mimics a variable resistor. In some embodiments of the EMR delivery system 20 a digital potentiometer may control the EMR output 24 of one or more EMR sources 22.

As described above the EMR source(s) 22 may be of several different types, each with its advantages. The EMR source(s) may be internal or external to the wound dressing 14. In some embodiments of the wound dressing assembly 10, the EMR source 22 may be an array 44 of LEDs (or other light sources) that deliver therapeutic EMR 24 (light) to wound area 32 tissue.

The microcontroller 94 is essentially is a small computer on an integrated circuit board capable of processing, and delivering a programmable input/output. The microcontroller 94 communicates with the other components of the electronic module 26 receiving feedback, analyzing that feedback, and executing tasks as programmed.

The sensors 96 may include temperature or pressure sensors, as well as other vital medical sensors that determine heart rate, pulse, oxygen saturation, etc. The feedback from the sensors 96 may be displayed on a monitor for a medical practitioner to review and evaluate to determine treatment efficacy and/or to determine if the treatment needs to be adjusted so that any adjustment may be programmed into the microcontroller 94.

The display/user interface 98 may include any display of power, timing, or treatment cycle. The display/user interface 98 may enable a user to turn on or off the unit, read recordings from any of the various sensors 96, and program the microcontroller 94. The display/user interface 98 may take several different forms. It may be as simple as the ON switch 52 and OFF switch 54 as described above, or it may enable selective and/or incremental increases/decreases in EMR output 24 intensity or selective and/or incremental increases/decreases in frequency of the light emitted by a slide switch, a dial or the like, or it may control may be provided to control any of the characteristics mentioned herein. Also, the display/user interface 98 may comprise a remotely located monitor (not shown) that visually displays pertinent data received from the microcontroller 94 regarding treatment parameters and information received from the sensors 96 and a keyboard (not shown) from which treatment parameters may be altered by programming the microcontroller 94. Where at least a portion of the display/user interface 98 is remotely located, the communication channel between the microcontroller 94 and the remote portion of the display/user interface 98 may be hard wired or wireless.

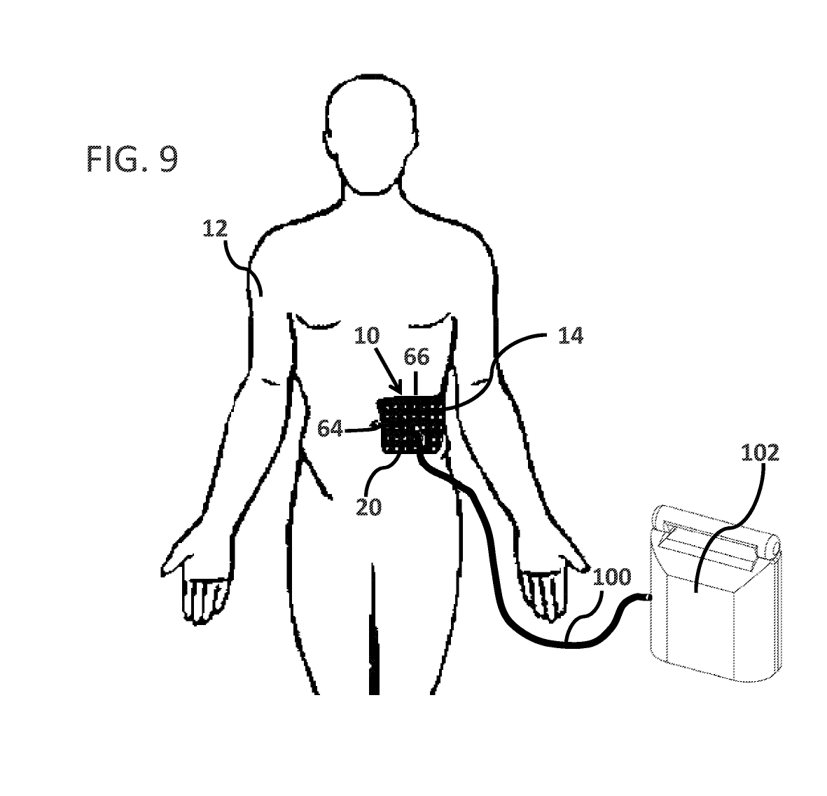

FIG. 9 is a schematic view of an exemplary wound dressing assembly 10 as applied to a patient showing the application of negative-pressure therapy through a pressure conduit 100 placed through the wound dressing 14 and connected to a remotely located vacuum pump 102. With this exemplary embodiment, the EMR delivery system 20 is self-contained within the wound dressing 14 and the pressure conduit 100 is disposed through the aperture 64 much like the drainage tube 32 of FIG. 1, except the pressure conduit 100 is sealed against the wound dressing 14 and the periphery 66 of the wound dressing is sealed against the patient 12 so not to compromise the application of negative pressure to the wound area 32. The EMR delivery system 20 and the vacuum pump 102 of this exemplary embodiment may operate independently of each other, simultaneously, or overlappingly.

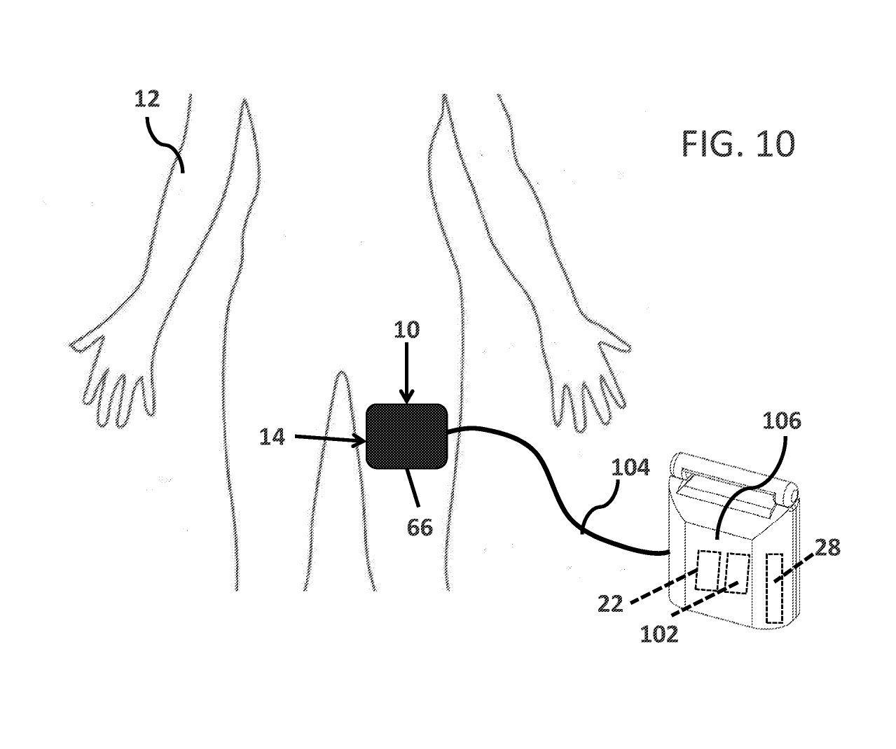

FIG. 10 is a schematic view of an exemplary wound dressing 10 as applied to a patient showing the combination application of therapeutic EMR and negative-pressure therapy through a delivery conduit 104 connected to a remotely located EMR source/vacuum pump combination unit 106. With this exemplary embodiment, at least a portion of the EMR delivery system 20 is remotely located and housed within the EMR source/vacuum pump combination unit 106 and the wound dressing 14 integrates with the delivery conduit 104 to facilitate the supply of both negative pressure and EMR output 24. The delivery conduit 104 is sealed against the wound dressing 14 and the periphery 66 of the wound dressing is sealed against the patient 12 so not to compromise the application of negative pressure to the wound area 32. In some embodiments, the delivery conduit 104 may serve a light column through which therapeutic EMR 24 travels down the delivery conduit 104 from an EMR source 22 within the EMR source/vacuum pump combination unit 106 to emit onto the wound area 32. In other embodiments, the delivery conduit 104 may serve to house fiber optics through which therapeutic EMR 24 propagates down the delivery conduit 104 from an EMR source 22 within the EMR source/vacuum pump combination unit 106 to emit onto the wound area 32. In yet other embodiments, the delivery conduit 104 may serve to house wires that supply power to EMR source(s) 22 disposed within the wound dressing 14, the wires being connected to a power supply 28 disposed within the EMR source/vacuum pump combination unit 106. Again, though disposed within the EMR source/vacuum pump combination unit 106, the EMR delivery system 20 and the vacuum pump 102 of these exemplary embodiments may operate independently of each other, simultaneously, or overlappingly.

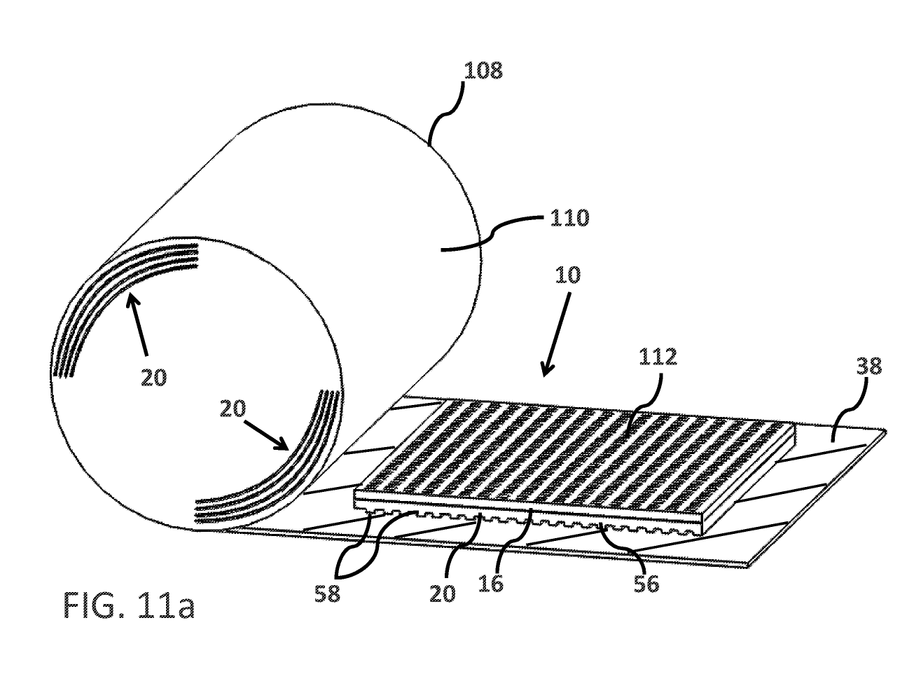

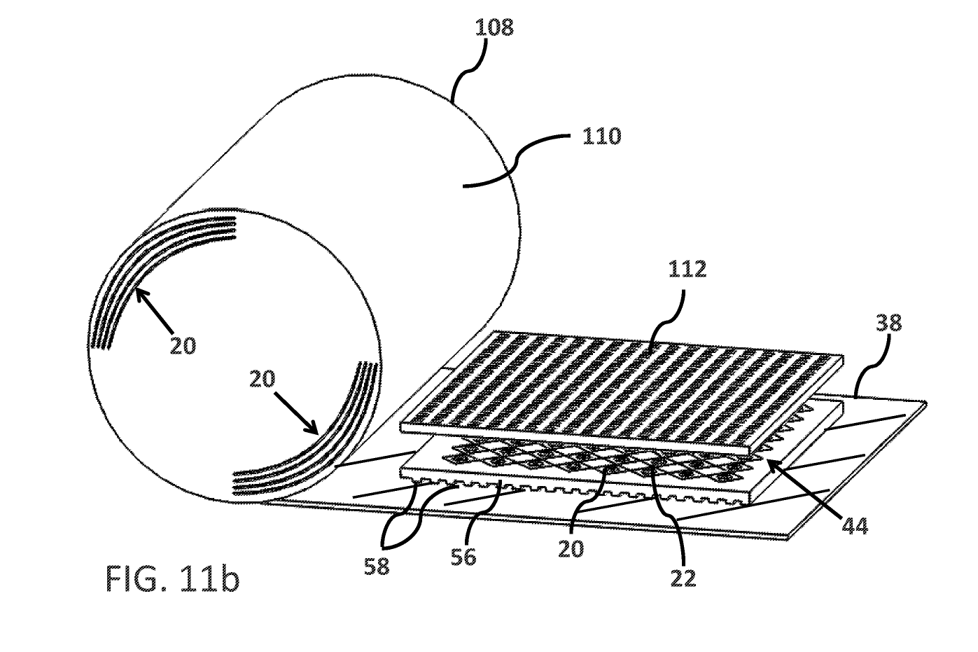

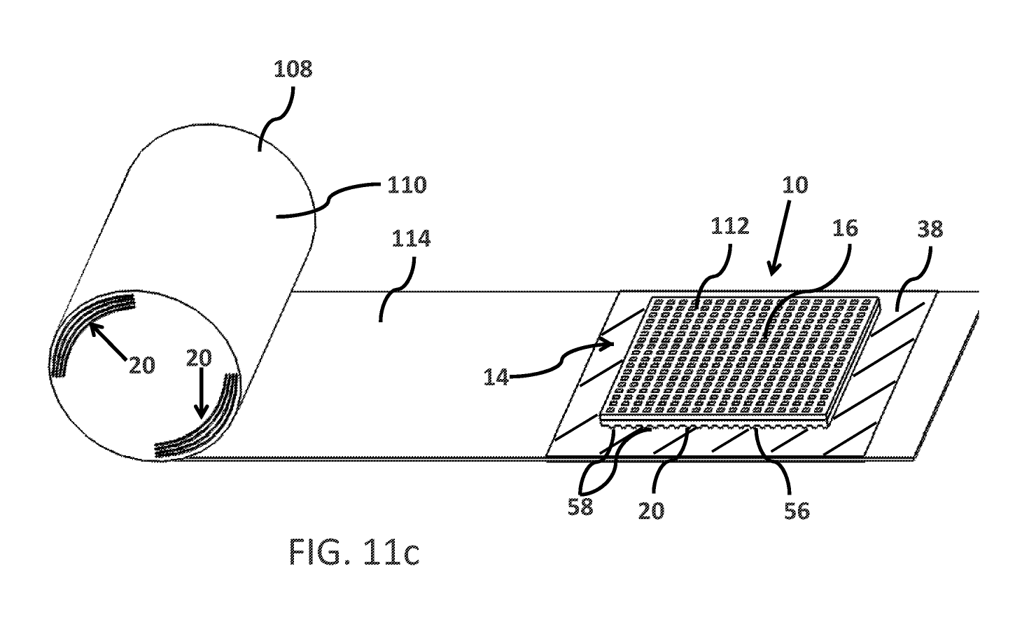

FIG. 11a is a perspective view of another exemplary embodiment of a wound dressing assembly 10 showing a roll 108 of wound dressings 14 capable of wrapping around a body part. The roll 108 of wound dressings 14 comprises a compression bandage wrap 110, an inner adhesive layer 38, a combination EMR delivery system 20/heat dissipating layer 56 where the EMR delivery system 20 is disposed on top of the heat dissipating layer 56 with the ridges 58 disposed on bottom, and a gauze bandage layer 112 serving as the optical layer 16. The inner adhesive layer 38 may have a non-stick film that overlays the inner adhesive layer 38 so that the adhesive will not stick to adjacent layers when rolled. The non-stick film may be removed to reveal the adhesive when the wound dressing is ready for application to a patient 12. Although wound dressing assemblies 10 may be packaged in individual packages, the roll 108 embodiment enables multiple wound dressing assemblies 10 to be packaged together in a single package which has storage advantages. Each EMR delivery system 20 may be self-contained or there may be an input connection for providing power, for example, to the EMR delivery system 20 from a remote location. The gauze bandage layer 112 may serve as a diffuser 72. The gauze bandage layer 112 may also be detachable so that it may be removed and discarded when soiled. When the gauze bandage layer 112 is detached and discarded when soiled with biological fluids, it may be replaced with a fresh, sterile gauze bandage layer 112.

FIG. 11b is a perspective, partially exploded view of the exemplary embodiment roll 108 of wound dressings 14 of FIG. 11a. The roll 108 of wound dressings 14 comprises a compression bandage wrap 110, an inner adhesive layer 38, a combination EMR delivery system 20/heat dissipating layer 56 where the EMR delivery system 20 (with an array 44 of EMR sources 22) is disposed on top of the heat dissipating layer 56 with the ridges 58 disposed on bottom, and a gauze bandage layer 112 serving as the optical layer 16. The inner adhesive layer 38 may have a non-stick film that overlays the inner adhesive layer 38 so that the adhesive will not stick to adjacent layers when rolled. The non-stick film may be removed to reveal the adhesive when the wound dressing is ready for application to a patient 12.

FIG. 11c is a perspective view of an exemplary embodiment showing a roll 108 of wound dressings 14 as depicted in FIGS. 11a and 11b wherein the roll 108 is unrolled partially to show an extended non-adhesive tail 114 for wrapping around a body part. With embodiments wherein the wound dressing 14 may be rolled up within a roll 108 and unrolled to wrap around a body part, the bandage wrapping portion allows for additional compression of the wound area 32 to prevent bleeding while the therapeutic EMR 24 is active. The wrapping portions of the roll 108, including the non-adhesive tails 114, may be made of various materials including by way of example knitted elastic, rubber, nylon, cotton, polyester, gauze, resilient fabrics, or any combination thereof.

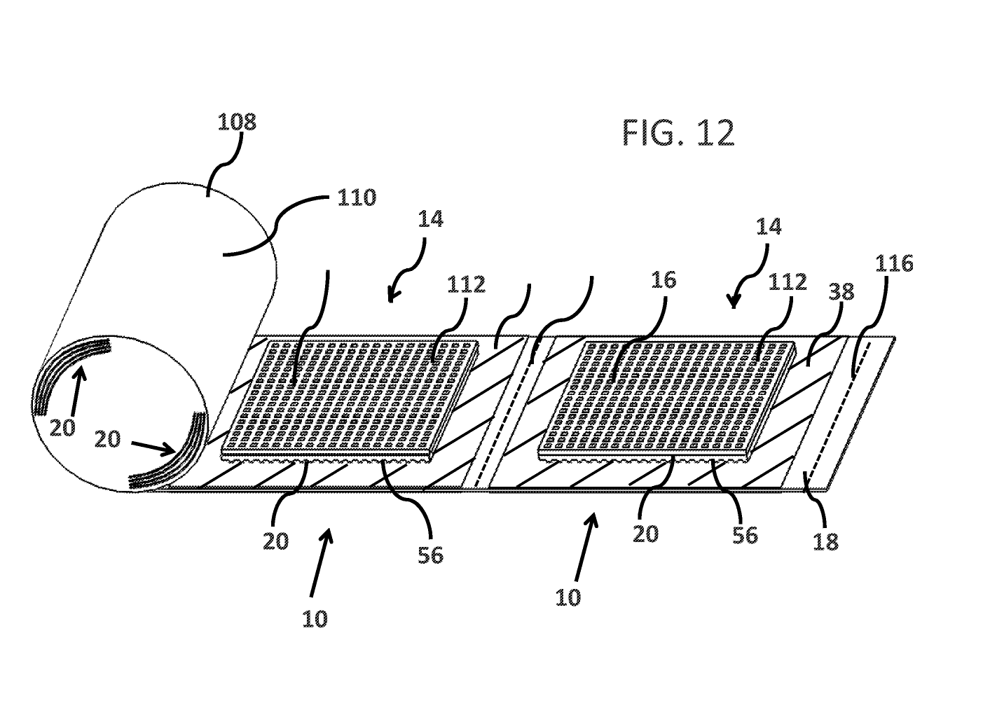

FIG. 12 is a perspective view of another exemplary embodiment showing multiple individual wound dressing assemblies 10 in a roll 108 for packaging purposes. Although similar to the embodiments depicted in FIGS. 11a, 11b, and 11c, this exemplary embodiment would not include the portion comprising the compression bandage wrap 110. Rather, this exemplary embodiment includes an inner adhesive layer 38 for each individual wound dressing 14, as well as a perforated strip 116 between wound dressings 14 to be torn away when separating a wound dressing 14 from the roll 108.

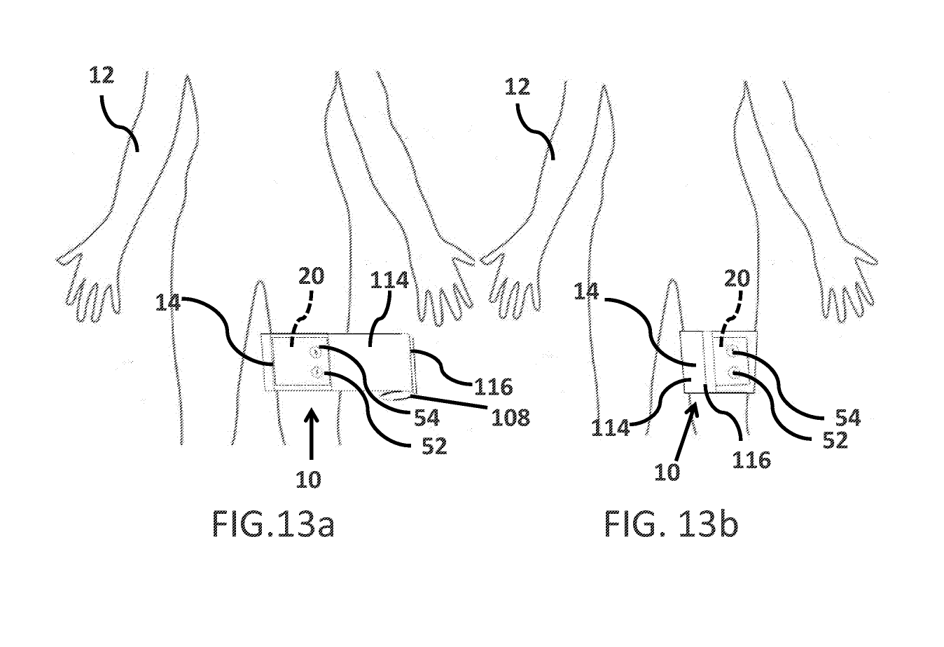

FIG. 13a is a schematic view of an exemplary wound dressing assembly 10 with self-contained controls and power source 48 (not visible) on a roll 108 showing an initial stage of as applying the exemplary wound dressing 14 to a patient 12. The self-contained controls depicted are the ON switch 52 and OFF switch 54 described above. As shown, the non-adhesive tail 114 ends in a perforated strip 116 that may be torn away when separating the wound dressing 14 from the roll 108 after wrapping the non-adhesive tail 114 around the body part.

FIG. 13b is a schematic view of the exemplary wound dressing assembly 10 with self-contained controls on a roll 108 of FIG. 13b showing the final stage of as applying the exemplary wound dressing 14 to a patient 12 so that the controls are not obscured. It should be noted that after the perforated strip 116 is torn away, if the non-adhesive tail 114 is too long and would obscure the self-contained controls, the length of the non-adhesive tail 114 may be trimmed as needed.

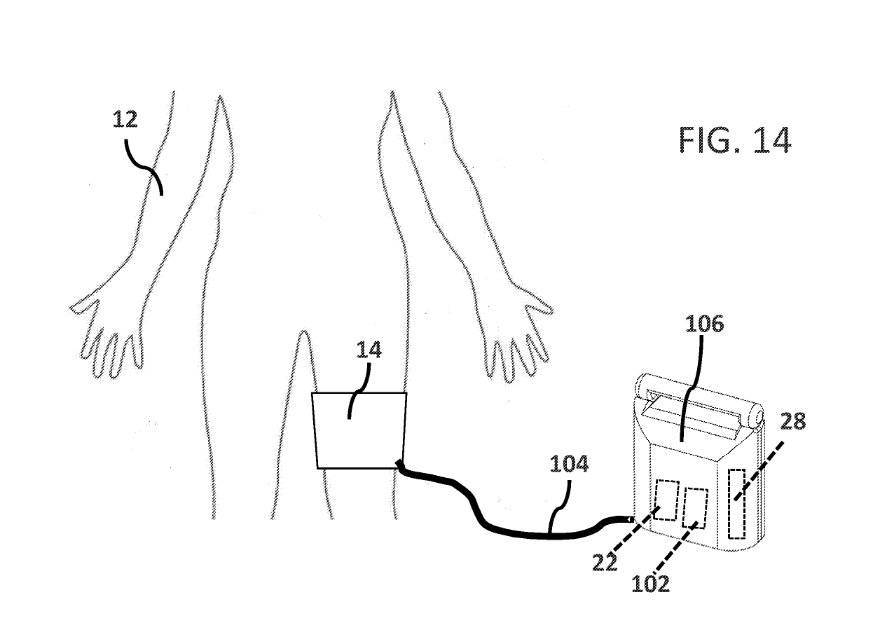

FIG. 14 is a schematic view of an exemplary wound dressing assembly 10 as wrapped around a patient 12 showing the combination application of therapeutic EMR 24 and negative-pressure therapy through a delivery conduit 104 connected to a remotely located EMR source/vacuum pump combination unit 106. With this exemplary embodiment, at least a portion of the EMR delivery system 20 is remotely located and housed within the EMR source/vacuum pump combination unit 106 and the wound dressing 14 integrates with the delivery conduit 104 to facilitate the supply of both negative pressure and EMR output 24. The delivery conduit 104 is sealed against the wound dressing 14 and the periphery 66 of the wound dressing is sealed against the patient 12 so not to compromise the application of negative pressure to the wound area 32. In some embodiments, the delivery conduit 104 may serve a light column through which therapeutic EMR 24 travels down the delivery conduit 104 from an EMR source 22 within the EMR source/vacuum pump combination unit 106 to emit onto the wound area 32. In other embodiments, the delivery conduit 104 may serve to house fiber optics through which therapeutic EMR 24 propagates down the delivery conduit 104 from an EMR source 22 within the EMR source/vacuum pump combination unit 106 to emit onto the wound area 32. In yet other embodiments, the delivery conduit 104 may serve to house wires that supply power to EMR source(s) 22 disposed within the wound dressing 14, the wires being connected to a power supply 28 disposed within the EMR source/vacuum pump combination unit 106. Again, though disposed within the EMR source/vacuum pump combination unit 106, the EMR delivery system 20 and the vacuum pump 102 of these exemplary embodiments may operate independently of each other, simultaneously, or overlappingly.

While specific embodiments and applications of the present invention have been illustrated and described, it is to be understood that the invention is not limited to the precise configuration and components disclosed herein. Various modifications, changes, and variations which will be apparent to those skilled in the art may be made in the arrangement, operation, and details of the methods and systems of the present invention disclosed herein without departing from the spirit and scope of the invention.

What is claimed is:

1. A flexible, therapeutic wound dressing assembly for placement on or in a patient, to absorb biological fluids, to protect a wound, and to deliver therapeutic electromagnetic radiation (EMR) to the patient, the therapeutic wound dressing assembly comprising:
a wound dressing wherein at least a portion of the dressing comprises an optical layer and an outer protective layer, the optical layer having at least an optical portion comprising at least one of an optically clear material, a translucent material, and semi-opaque material that allows the therapeutic EMR to pass through with minimal optical obstruction; and
an EMR delivery system comprising:
at least one EMR source that emits non-ultraviolet, therapeutic EMR having intensity sufficient to activate desired therapeutic properties within the patient;
at least one electronic module that controls EMR output from the at least one EMR source, the EMR output controlled comprising at least one of wavelength, intensity, fluence, frequency, duty cycle, and treatment pattern; and
a power supply for the EMR source.

2. A wound dressing assembly as in claim 1, wherein the at least one EMR source comprises an optical element, the optical element being selected from the group consisting of light emitting diodes, lasers, filtered fluorescents, filtered incandescents, electroluminescent wire, and any combination thereof.

3. A wound dressing assembly as in claim 1, wherein the therapeutic EMR is delivered at a predetermined duty cycle.

4. A wound dressing assembly as in claim 1, wherein the therapeutic EMR has at least one wavelength that ranges from about 380 nm to about 900 nm.

5. A wound assembly as in claim 4, wherein the at least one wavelength of therapeutic EMR comprises a predominant wavelength selected to sterilize one or more target organisms, the predominant wavelength is selected from a group of wavelengths consisting of wavelengths centered about 400 nm, 405 nm, 415 nm, 430 nm, 440 nm, 455 nm, 470 nm, 475 nm, 660 nm, and 808 nm.

6. A wound dressing assembly as in claim 5, wherein the predominant wavelength alternates between a first predominant wavelength and a second predominant wavelength in a selected treatment pattern.

7. A wound dressing assembly as in claim 1, wherein the at least one EMR source comprises a first EMR source for delivering a first therapeutic EMR to a first location on the patient and a second EMR source for delivering a second therapeutic EMR to a second location on the patient, the first therapeutic EMR being different from the second therapeutic EMR, the first location being different from the second location.

8. A wound dressing assembly as in claim 1, further comprising a diffuser that diffuses the therapeutic EMR such that the therapeutic EMR is distributed relatively evenly over a surface area covered by the wound dressing assembly.

9. A wound dressing assembly as in claim 1, wherein the at least one EMR source comprises a plurality of EMR sources wherein each EMR source is arranged in an array and is spaced from each other of the EMR sources such that the therapeutic EMR is distributed a surface area covered by the wound dressing assembly.

10. A wound dressing assembly as in claim 1, wherein the wound dressing further comprises a heat dissipation layer disposed proximate the at least one EMR source to provide a passive exchange of heat away from the patient.

11. A wound dressing assembly as in claim 1, wherein power density of the therapeutic EMR is within a range from 1.0 mW/cm² and 1.0 W/cm².

12. A wound dressing assembly as in claim 1, wherein the fluence of the therapeutic EMR is within a range from 1.0 mJ/cm² and 1.0 kJ/cm².

13. A wound dressing assembly as in claim 1, wherein the therapeutic EMR is pulsed at a rate between 0 Hz and 5,000 Hz and at a pulse width between 10 nanoseconds and 1 second.

14. A wound dressing assembly as in claim 1, wherein the wound dressing is flexible and adhered to a wrapping roll, the wrapping roll being comprised of a material from the group of materials consisting of cloth, gauze, a woven fabric, a synthetic fabric, and an elastic fabric.

15. A wound dressing assembly as in claim 1, wherein the outer layer comprises an activation switch.

16. A wound dressing assembly as in claim 1, wherein at least a portion of the EMR delivery system is detachable from the therapeutic wound dressing such that a soiled therapeutic wound dressing may be discarded and a fresh therapeutic wound dressing is an attachable replacement and the EMR delivery system is reusable.

17. A flexible, therapeutic wound dressing assembly for placement on or in a patient, to absorb biological fluids, to protect a wound, and to deliver therapeutic electromagnetic radiation (EMR) to the patient, the therapeutic wound dressing assembly comprising:
a wound dressing wherein at least a portion of the dressing comprises an optical layer, an outer protective layer, and an aperture through the optical layer and the outer protective layer, the optical layer having at least an optical portion comprising at least one of an optically clear material, a translucent material, and semi-opaque material that allows the therapeutic EMR to pass through with minimal optical obstruction, the aperture for receiving one of a drainage tube, a catheter, a medical sensor, and an electrostimulation probe; and
an EMR delivery system comprising:
at least one EMR source that emits non-ultraviolet, therapeutic EMR having intensity sufficient to activate desired therapeutic properties within the patient;
at least one electronic module that controls EMR source output, the EMR source output controlled comprising at least one of wavelength, intensity, fluence, frequency, duty cycle, and treatment pattern; and
a power supply for the EMR source.

18. An assembly as in claim 17, wherein the wound dressing further comprises a periphery and a frangible line extending from the aperture to the periphery of the wound dressing.

19. A flexible, therapeutic wound dressing assembly for placement on or within a patient, to remove biological fluids, to protect damaged tissue, and to deliver therapeutic electromagnetic radiation (EMR) to the patient, the therapeutic wound dressing assembly comprising:
a wound dressing wherein at least a portion of the dressing comprises an optical layer and an outer protective layer, the optical layer having at least an optical portion comprising at least one of an optically clear material, a translucent material, and semi-opaque material that allows the therapeutic EMR to pass through with minimal optical obstruction; and
a vacuum pump for delivering negative-pressure to damaged tissue; and
an EMR delivery system comprising:
at least one EMR source for emitting non-ultraviolet, therapeutic EMR having intensity sufficient to activate desired therapeutic properties within the patient;
at least one electronic module capable of controlling and modulating at least one of EMR source output, wavelength, intensity, fluency, frequency, duty cycle, and treatment pattern; and
a power supply for the EMR source.

20. A wound dressing assembly as in claim 19, wherein at least a portion of the therapeutic EMR delivery system is remote from the wound dressing and is connected to the wound dressing with at least one of electrical wires and fiber optics.
